(19) United States
(12) Patent Application Publication
(10) Pub. No.: US 2011/0224763 A1
(43) Pub. Date: Sep. 15, 2011

(54) Apparatus and Device for Spot Heat Treating Skin

(76) Inventor: Galina Podolskiy, Chicago, IL (US)

(21) Appl. No.: 13/025,710

(22) Filed: Feb. 11, 2011

Related U.S. Application Data
(60) Provisional application No. 61/303,430, filed on Feb. 11, 2010.

(51) Int. Cl. A61F 7/08

(52) U.S. Cl. 607/114

(57) ABSTRACT

A heating device is provided for treating skill related conditions, comprising a power source, a heating element connected to the power source, and an outer shell that encases the heating element, the outer shell being made of wood.

### CROSS REFERENCE TO RELATED APPLICATIONS

The present application claims the benefit of U.S. Provisional Application No. 61/303,430, filed Feb. 11, 2010, entitled, “Apparatus and Device for Spot Heat Treating Skin”, herein incorporated by reference.

### BACKGROUND

The present invention relates to a heating device and method used to treat cold sores and other spot heat-treatable skin infirmities, infections, and the like.

### SUMMARY

A heating device is provided for treating skill related conditions, comprising: a power source; a heating element connected to the power source; and an outer shell that encases the heating element, wherein the outer shell is made of wood.

### BRIEF DESCRIPTION OF THE DRAWINGS

The invention is described in more detail below, with reference to the embodiments shown in the drawings and the following description.

FIG. 1 is a cross-sectional view of a device for spot heat treating the skin according to an embodiment of the invention;

FIG. 2 is a cross-sectional view of a device for spot heat treating the skin according to another embodiment of the invention;

FIG. 3A is a cross-sectional view of a device for spot heat treating the skin according to another embodiment of the invention; and

FIG. 3B is a top view of the embodiment shown in FIG. 3A.

### DETAILED DESCRIPTION OF THE EMBODIMENTS

The various embodiments of the invention are described below.

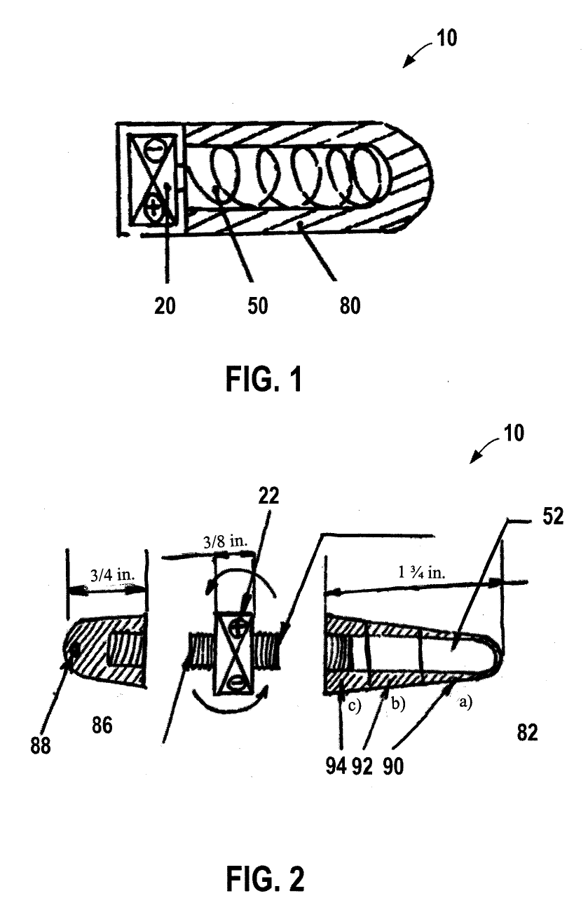

FIG. 1 is a cross section of an exemplary heating device 10 that can be used to treat cold sores and the like by applying heat to a specific point on the body. As can be seen in FIG. 1, the device 10 comprises a power component 20. This power component 20 is preferably a battery or other form of power storing device. In one embodiment, the power component 20 could comprise a plug so that it may be plugged into a wall during use—in this case the power component 20 could also provide power conditioning circuitry, such as a rectifier and voltage transformers.

The device 10 also comprises a heating unit 50 that is attached to the power component 20 and generate heat. The heating unit 50 can be any form of element that can generate sufficient heat after being applied power. Such a heating unit could include, e.g., nichrome wire, semiconductors, electrically resistive elements and the like.

The device 10 further comprises a shell assembly 80 that, in a preferred embodiment is made of wood due to the conductive properties of many types of wood. This wooden shell 80 conducts heat from an internal heating unit 50 to the skin as a direct contact device. In a simple design of the device 10, there is no feedback or control mechanism that is used to limit the heat. Rather, the design of the power component 20 in conjunction with the heating element 50 could be used to limit the heat. For example, if a 3V nickel-cadmium battery having an internal resistance of, e.g., 0.075Ω is used as the power component 20, and a 4Ω heating element 50 resistance is used, then the heating element 50 can produce approximately 2.2 W. Although this design does not regulate the heat precisely, the use of precision components and manufacture can be used to produce a reasonable range of device temperatures. However, this design would not be able to accommodate, e.g., the voltage fall-off as the battery becomes discharged.

A control mechanism may be implemented that precisely controls the temperature by e.g., turning a heat element on and off or introducing additional resistance in the circuit so that an optimum temperature with little variation can be applied to the skin without causing burns while still being effective. Known sensors, such as thermocouples and the like, can be provided to measure the temperature and provide input to a regulator that may include a processor or simpler comparative circuitry with a reference component. In this way, much more precise temperature control can be realized.

A temperature of the outer surface of the device 10 should be kept at some maximum temperature that will not burn the skin (around 130° F.). This is because such heat is effective to destroy the virus that causes cold sores or other forms of viral or bacterial infections. The device 10 could also have a user adjustable element so that the user could adjust the maximum temperature of the device to a temperature that suits them.

FIG. 2 illustrates an embodiment and some exemplary measurements of the device 10. As can be seen, a battery as a power supply can be provided in a battery compartment 22, which may be flippable (i.e., pivotable) in order to provide a way of turning the device off without requiring a switch. In this way, it can be assured that the device is not turned on when it is put away for storage. The heating element 52 is located in an area such that the heat can be conducted to the outer surface of the shell assembly 80. The small size and portability means that the device can be applied quickly after noticing symptoms, such as burning or itching skin, rather than after, e.g., a cold sore is noticed (since it can be more difficult to get rid of later).

The shell assembly is designed to have a front 82 and rear 86 part. The rear part 86 may have a hole 88 so that it can be used on a key chain or other holding device easily.

In a preferred embodiment, the shell front 82 is designed so that it is thinner on the tip than on the sides. This permits a varying degree of temperatures to be present on the device, and colored rings 90,92 94, or other form of identifying indicia may be used to indicate approximate temperature ranges. For example the first region 90 (a) may indicate a temperature in the range of 125-130° F., whereas the second region 92 (b) may indicate a temperature in the range of 115-125° F., and the third region 94 (c) may indicate a temperature in the range of 105-115° F. In this way, a user could press different areas of the device against the skin, depending on the temperature that works best.

In a very general sense, the device 10 is the size of a finger or smaller. This permits it to be carried in the cosmetic case or the pocket, or to become a key chain. As illustrated in FIG. 2, the device could have a total length of approximately 2.5-3.0″, and a diameter of possibly 0.75-1.0″. These are general sizes according to a preferred embodiment—however, other sizes are to be considered within the scope of the invention.

The use of wood for the shell assembly 80 is provided in a preferred embodiment because of its conductive properties. It is not completely a thermal conductor, as metals are, nor is it a complete insulator that would limit the ability of heat to reach the outer surface. Furthermore, since wood is combustible at over 300° F., it would not be damaged by the heating element that is controlled in a proper way, although it might be possible to include a metallic portion on the inside of the device. Porous types of wood are best for better heat conduction, including, e.g., basswood, birch, walnut, cherry, buckeye, maple, poplar, and willow.

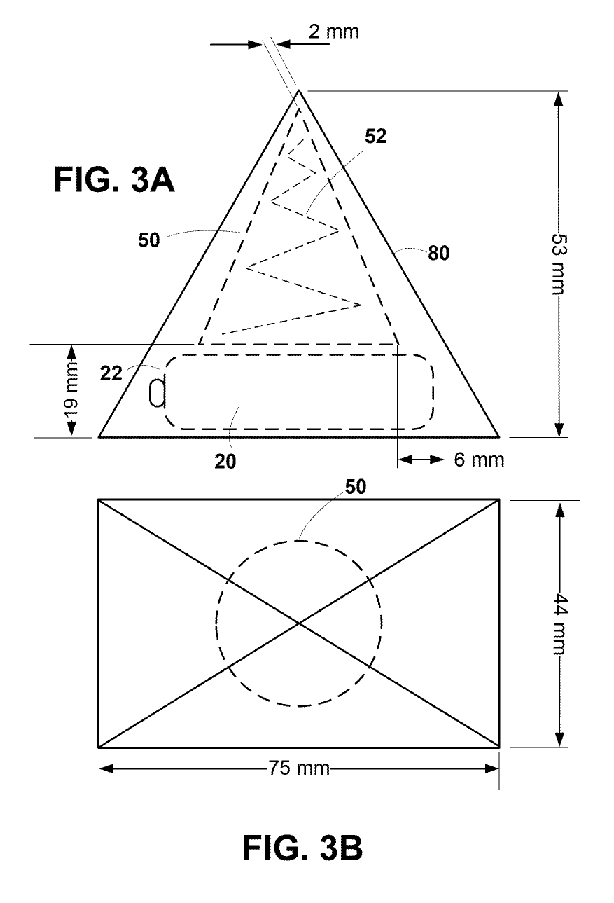

FIGS. 3A and 3B show an alternate embodiment in which the device has a pyramid shape. Although the device is shown as an elongated five-sided pyramid, it could have any number of sides and can also be symmetrical. As can be seen, a heating unit compartment 50, which is preferably made of ceramic, with a heating element 52 is located in a compartment in a top part of the pyramid. The device further has a battery compartment 22 for holding a battery or other power component 20 in a lower area. In a preferred embodiment, the battery is a nine-volt rechargeable battery. The dimensions provided in these figures are exemplary, but it can be seen that the thickness of the pyramid walls vary from being thinnest at the top and thickening towards the bottom of the pyramid.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, processing elements, and the like, which may carry out a variety of functions under the control of one or more control devices. The present invention could employ any number of conventional techniques for electronics configuration, signal processing and/or control, data processing and the like.

The particular implementations shown and described herein are illustrative examples of the invention and are not intended to otherwise limit the scope of the invention in any way. For the sake of brevity, conventional electronics, control systems, and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”.

The use of the terms “a” and “an” and “the” and similar referents in the context of describing the invention (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Finally, the steps of all methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. The words “mechanism” and “element” are intended to be used generally and are not limited solely to mechanical embodiments. Numerous modifications and adaptations will be readily apparent to those skilled in this art without departing from the spirit and scope of the present invention.

### DESCRIPTION OF REFERENCE CHARACTERS

10 heating device

20 power component

22 battery compartment

50 heating unit

52 heating element

80 shell assembly

82 shell front

86 shell rear

88 hole

90, 92, 94 colored rings to indicate temperature

What is claimed is:

1. A heating device for treating skill related conditions, comprising:
a power source;
a heating element connected to the power source; and
an outer shell that encases the heating element, wherein the outer shell is made of wood.

2. A heating device for treating skill related conditions, comprising:
a power source;
a heating element connected to the power source; and
an outer shell that encases the heating element, wherein the outer shell has a thickness that varies in thickness, being thinnest at a tip end and thicker away from the tip end to produce a temperature gradient from the tip end towards a direction away from the tip end.

3. The heating device of claim 2, wherein a temperature range in the different areas varies from approximately 105° F. to 130° F.

4. The heating device of claim 2, further comprising identifying indicia to indicate approximate temperature ranges.

5. The heating device of claim 2, having an elongated circular cross-sectional shape.

6. The heating device of claim 2, having a pyramidal shape.

7. The heating device of claim 2, further comprising an outer shell that encases the heating element, wherein the outer shell is made of wood.

8. The heating device of claim 7, wherein the wood is selected from the group consisting of basswood, birch, walnut, cherry, buckeye, maple, poplar, and willow.

9. The heating device of claim 2, wherein the heating element is an electrically resistive element.

10. The heating device of claim 9, wherein the heating element is either nichrome wire or a semiconductor.

11. The heating device of claim 2, wherein the heating element is at least partially within a ceramic element.

12. The heating device of claim 2, further comprising a temperature regulator.

13. A heating device for treating skill related conditions, comprising:
a heating element;
a power source that is connected to the heating element when the power source is on an operating condition, and is disconnected from the heating element when the power source is in a storage position; and
an outer shell that encases the heating element.
